(19) United States
(12) Patent Application Publication
(10) Pub. No.: US 2024/0101989 A1
(43) Pub. Date: Mar. 28, 2024

(54) NUCLEASE-MEDIATED GENOME EDITING

(71) Applicant: Wageningen Universiteit, Wageningen (NL)

(72) Inventor: John VAN DER OOST, Renkum (NL)

(73) Assignee: Wageningen Universiteit, Wageningen (NL)

(21) Appl. No.: 18/461,941

(22) Filed: Sep. 6, 2023

Related U.S. Application Data
(62) Division of application No. 17/554,465, filed on Dec. 17, 2021, now Pat. No. 11,760,985.
Application No. 17/554,465 is a continuation of application No. 16/400,021, filed on Apr. 30, 2019, now Pat. No. 11,479,761.
Application No. 16/400,021 is a continuation of application No. 15/566,528, filed on Oct. 13, 2017, now Pat. No. 11,053,482.
(60) Provisional application No. 62/269,143, filed on Dec. 18, 2015.
(60) Provisional application No. 62/312,724, filed on Mar. 24, 2016.

(30) Foreign Application Priority Data
Apr. 16, 2015 (GB) 1506509.7

(51) Int. Cl. C12N 9/22; C12N 15/11

(52) U.S. Cl. CPC C12N 9/22; C12N 15/11; C07K 2319/09; C12N 2310/20

(57) ABSTRACT

The invention related to the field of genetic engineering tools, methods and techniques for gene or genome editing. Specifically, the invention concerns isolated polypeptides having nuclease activity, host cells and expression vectors comprising nucleic acids encoding said polypeptides as well as methods of cleaving and editing target nucleic acids in a sequence-specific matter. The poly peptides, nucleic acids, expression vectors, host cells and methods of the present invention have application in many fields of biotechnology, including, for example, synthetic biology and gene therapy.

### CROSS-REFERENCE TO RELATED APPLICATIONS

This application is a Divisional of U.S. application Ser. No. 17/554,465, filed Dec. 17, 2021, which is a Continuation of U.S. application Ser. No. 16/400,021, filed Apr. 30, 2019, now U.S. Pat. No. 11,479,761 that issued on Oct. 25, 2022, which is a Continuation of U.S. application Ser. No. 15/566,528, filed Oct. 13, 2017, now U.S. Pat. No. 11,053,482, that issued on Jul. 6, 2021, which is the National Phase of International Patent Application No. PCT/EP2016/058442, filed Apr. 15, 2016, published on Oct. 20, 2016 as WO 2016/166340 A1, which claims priority to United Kingdom Patent Application No. 1506509.7, filed Apr. 16, 2015, U.S. Provisional Application No. 62/269,143, filed Dec. 18, 2015 and U.S. Provisional Application No. 62/312,724, filed Mar. 24, 2016. The contents of these applications are herein incorporated by reference in their entirety.

### SEQUENCE LISTING

The instant application contains a Sequence Listing which has been submitted in XML format and is hereby incorporated by reference in its entirety. Said XML copy, created on Dec. 17, 2023, is named 114203_6100_SL.xml and is 61,382 bytes in size.

### FIELD OF THE INVENTION

The invention relates to the field of genetic engineering tools, methods and techniques for genome or gene editing. Such editing or manipulation of polynucleotide sequences, including structural or control gene sequences has application in many fields of health and biotechnology, for example gene therapy treatments of humans or animals, plant and animal breeding, and improvement of industrial organisms, e.g. by altering enzymes and metabolic pathways, particularly microorganisms; also in the areas of synthetic biology and algal biofuel production for example. Also the invention further relates to research tools and methods for use in basic scientific research involving molecular genetics.

### BACKGROUND TO THE INVENTION

Site-specific nucleases can permit the generation of double strand breaks (DSBs) at selected positions along a strand of DNA. In an organism of interest, this enables DSBs to be made at pre-determined positions in the genome. The creation of such breaks by site-specific nucleases prompts the endogenous cellular repair machinery to be repurposed in order to insert, delete or modify DNA at desired positions in the genome of interest. Targeted DNA cleavage mediated by site-specific nucleases is therefore an important basic research tool which has facilitated the functional determination and annotation of specific genes but amongst other things has also enabled the targeted mutation, addition, replacement or modification of genes in organisms of agricultural, industrial or commercial significance. As the genetic basis of both desirable and undesirable organismal phenotypes is uncovered through DNA sequencing, the ability to generate targeted alterations at specific genomic loci is fundamental to the genetic engineering of useful traits and in the development of clinical treatments for diseases with a genetic basis.

Other site specific nuclease approaches involve single strand target nucleic acid breaks, whether singly or in combination.

During the past decade, a range of molecular tools have been developed to allow for specific genetic engineering in general, and for dedicated editing of eukaryotic genomes in particular. Initially Zinc-Finger Nucleases (ZFNs) were developed, followed by Transcription Activator-Like Effector Nucleases (TALEN5). Recently, a revolution has been caused by the development of the CRISPR-associated Cas9 nuclease, as a very efficient, generic and cheap alternative for dedicated genome surgery in a range of eukaryotic cells (from yeast and plant to zebrafish and human) (reviewed by Van der Oost 2013, Science 339: 768-770, and Charpentier and Doudna, 2013, Nature 495: 50-51).

Many useful site-specific nucleases have been discovered in and isolated from prokaryotes. Just like eukaryotes, prokaryotic organisms possess a variable set of defence systems to protect themselves against viruses. The defence strategies that protect their microbial host against invading DNA mainly rely on general (innate) immunity systems, such as the well-known restriction enzymes.

A major recent discovery in this area has been the demonstration of a specific (adaptive) immunity system in bacteria and archaea. This adaptive immune system consists of clustered regularly interspaced palindromic repeats (CRISPR), and CRISPR-associated Cas genes that encode the Cas proteins. The CRISPR-Cas system uses small CRISPR RNAs that guide effector Cas proteins to complementary invading nucleic acids, eventually neutralizing the invasion. Two classes of Cas effector complexes are distinguished: multi-subunit complexes (e.g. E. coli Cascade) and single-protein systems (e.g. Streptococcus pyogenes Cas9) (Van der Oost et al., 2014, Nature Rev. Microbial. 12: 479-492).

Molecular analyses of CRISPR-Cas have provided the foundation for the development of genome engineering tools. Cas9 is a relatively simple CRISPR-Cas effector complex that can be functionally expressed in a wide range of prokaryotic and eukaryotic cells. Importantly, the RNA guide of Cas9 can easily be manipulated to specifically target any sequence of interest. Although adjusting the specificity for a certain target gene is also possible with the TALEN system, a drawback of this system is that this requires laborious protein engineering. In case of Cas9, only a short oligonucleotide has to be generated and cloned, saving time and money. Applications of the Cas9 system include general genetic engineering (disruption, repair and integration of genes), control of gene expression (stimulation and silencing) and gene labelling (imaging). Co-expression of Cas9 with different guides allows for multiplexing, for instance generating multiple knockouts simultaneously.

The CRISPR-Cas system allows target-specific cleavage of genomic DNA guided by Cas9 nuclease in complex with a guide RNA (gRNA) that complementarily binds to a 20 nucleotide targeted sequence. Alteration of the sequence of the gRNA therefore allows the Cas9 endonuclease to be programmed to cut double-stranded DNA at sites complementary to the 20-base-pair guide RNA. The Cas9 system has been used to modify genomes in multiple cells and organisms.

Compared with alternative genome editing systems (Zinc Finger Nucleases, TALEN), engineering by Cas9 is very efficient, cheap, and fast.

Despite these developments, the Cas9 system still has some practical draw-backs. Firstly, based on an intrinsic self/non-self-discrimination mechanism, Cas9 requires a sequence motif (protospacer adjacent motif, PAM) in the flanking region adjacent to the target sequence. The PAM-requirement imposes a significant design limitation on the endonuclease system, excluding potential target sites.

Secondly, although RNA-guided nucleases such as Cas9 incorporate guide RNAs which direct cleavage of specific target sites and therefore exhibit a reduction in the significant off-target activity observed in most other available nucleases, a certain level of off-target cleavage still occurs (Pattanayak et al., 2013, Nat. Biotechnol. 31: 839-843), that is, cleavage of genomic sequences that differ from the intended target sequence by one or more nucleotides. Generally, 15-17 nucleotides are required for base pairing with a 20 nucleotide complementary target; the tolerance for mismatches having been hypothesized to explain reported off-target problems. The imperfect specificity of engineered site-specific binding can lead to unintended insertion, modification or deletion of genomic loci during a gene targeting event, which has been associated with cellular toxicity. The consequences of such off target cleavage events resulting in undesired alterations of genomic loci other than the desired target can be extremely serious in a clinical context.

The sequence-specific cleavage of the intended nuclease target site in the absence of, or with only minimal background off-target cleavage activity is a prerequisite for high-efficiency genomic manipulation in basic research applications and especially in avoiding the cleavage of unintended genes during targeted genomic modifications associated with clinical applications of the site-specific endonuclease technologies, particularly since the resulting double-stranded breaks result in stable, heritable genome modifications.

Despite a great deal of attention being focussed on addressing these undesired features of the Cas9 system, to date they remain largely unresolved.

Imprecise specificity in particular continues to remain a difficulty and has only partially been addressed by expanding the to-be-recognised target sequence by dimers of catalytically inactivated Cas9 fused to the nuclease domain of Fokl (dCas9-Fokl) (Guilinger et al., 2014, Nat. Biotechnol. 32: 577-582). In addition, engineered nickase variants of Cas9 (in which one of the two nuclease sites is disrupted) have been demonstrated to facilitate homology directed repair in eukaryotic genomes with increased specificity and reduced off-target activity (Ran et al., 2013, Cell 154: 1380-1389. Also, Mali et al., 2013, Nat. Biotechnol. 31: 833-838).

WO 2015/035139 describes compositions, methods, systems, and kits for controlling the activity and/or improving the specificity of RNA-programmable endonucleases, such as Cas9. For example, guide RNAs (gRNAs) are engineered to exist in an “on” or “off” state, which control the binding and hence cleavage activity of RNA-programmable endonucleases. Also described are mRNA-sensing gRNAs that modulate the activity of RNA-programmable endonucleases, based on the presence or absence of a target mRNA. Some gRNAs are described that modulate the activity of an RNA-programmable endonuclease based on the presence or absence of an extended DNA (xDNA).

Another approach to mitigate off-target activity has centred on the development of software packages to aid in the guide RNA design process by undertaking exhaustive target sequence searches against genomic reference sequences, allowing the selection of target sequences with minimal off-target cleavage effects (Naito et al., 2015, Bioinformatics 31: 1120-1123). However, this merely enables efficient exploration of the target sequence space available for guide sequence design rather than directly addressing the inherent limitations of CRISPR-Cas9 as a genome editing tool.

Thus, currently available nucleases, including CRISPR-Cas9 systems, are not in their current state of development necessarily suitable for the majority of clinical applications or indeed many other target-sensitive genome editing applications. There is a continuing need for genome editing tools with greater inherent specificity and reliability than is currently available in the art.

Schunder et al. provided the first indication of a functional CRISPR/Cas system in Francisella tularensis (Schunder et al., 2013, International Journal of Medical Microbiology 303: 51-60). However, until now the structure and functionality of the system has remained unclear.

Subsequently, a classification of all known CRISPR adaptive immune systems of Archaea based primarily on their concatenated Cas protein sequences was provided by Vestergaard et al. in which Cas_Cpf1 was identified as a single protein interference system lacking Cas3, Cas5, Cas7 and Cas8, reminiscent of Cas9 in bacterial Type II systems despite not appearing to share any structural domains (Vestergaard et al., 2014, RNA biology 11.2 {2014): 156-167).

### SUMMARY OF THE INVENTION

In seeking to overcome certain practical disadvantages associated with the Cas9 systems, the inventors provide a novel nuclease (Cpf1) unrelated to Cas9 for application as a gene editing tool. Cpf1 has been found to have uniquely advantageous mechanistic features such as a single nuclease domain and an upstream PAM motif and finds application as an improved tool for dedicated genome editing in general, and for repairing genetic disorders of human stem cells. Additionally, the Cpf1 nuclease can function as part of a multiplex engineering system for micro-organisms.

Accordingly, the present invention provides an isolated polypeptide or fragment thereof, comprising the amino acid sequence SEQ ID NO: 1 or a sequence of at least 60% identity therewith, and having a nuclease activity.

In preferred aspects, the polypeptide or fragment comprises an amino acid sequence of at least 75%; preferably at least 85%; more preferably at least 90%; even more preferably at least 95% of SEQ ID NO:1.

The invention is based on reference SEQ ID NO:1 but includes any variant sequence having the defined percentage identity therewith. Such percentage identities include any of the following: a reference nucleic or amino acid sequence and sequences of at least a certain percentage identity are disclosed, e.g. at least 60%, then optionally the percentage identity may be different. For example: a percentage identity which is selected from one of the following: at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%. Such sequence identity with a SEQ ID NO: 1 amino acid sequence is a function of the number of identical positions shared by the sequences in a selected comparison window, taking into account the number of gaps, and the length of each gap, which need to be introduced for optimal alignment of the two sequences.

In all aforementioned aspects of the present invention, amino acid residues may be substituted conservatively or non-conservatively. Conservative amino acid substitutions refer to those where amino acid residues are substituted for other amino acid residues with similar chemical properties (e.g., charge or hydrophobicity) and therefore do not alter the functional properties of the resulting polypeptide. Similarly it will be appreciated by the skilled reader that nucleic acid sequences may be substituted conservatively or non-conservatively without affecting the function of the polypeptide. Conservatively modified nucleic acids are those substituted for nucleic acids which encode identical or functionally identical variants of the amino acid sequences. It will be appreciated by the skilled reader that each codon in a nucleic acid (except AUG and UGG; typically the only codons for methionine or tryptophan, respectively) can be modified to yield a functionally identical molecule. Accordingly, each silent variation (i.e. synonymous codon) of a polynucleotide or polypeptide, which encodes a polypeptide of the present invention, is implicit in each described polypeptide sequence.

The present invention provides a polypeptide or fragment having nuclease activity and comprising the amino acid sequence motif: FQIYN (SEQ ID NO: 2). This corresponds to residues 786-790 of SEQ ID NO:1.

The present invention also provides a polypeptide or fragment having nuclease activity and comprising the amino acid sequence motif: FQIYNK (SEQ ID NO: 3). This corresponds to residues 786-791 of SEQ ID NO:1.

The present invention also provides a polypeptide or fragment having nuclease activity and comprising the amino acid sequence motif: FQIYNKD (SEQ ID NO: 4). This corresponds to residues 786-792 of SEQ ID NO:1.

The present invention also provides a polypeptide or fragment having nuclease activity and comprising the amino acid sequence motif: X¹X²X³X⁴X⁵FQIYNKDX⁶X⁷ corresponding to residues 781-794 of SEQ ID NO:1, wherein X¹ is one of G or K, X² is one of K, S or D, X³ is one of L or I, X⁴ is one of Y or F, X⁵ is one of L or M, X⁶ is one of F or Y and X⁷ is one of S, A or V (SEQ ID NO: 5).

In another aspect the present invention provides a polypeptide or fragment having nuclease activity and comprising the amino acid sequence motif: GKLYLFQIYNKDFS (SEQ ID NO: 6). This corresponds to residues 781-794 of SEQ ID NO:1.

The amino acid sequence motif may instead comprise residues selected from 784-794, 785-794, 786-794, 787-794, 788-794 or 789-794 of SEQ ID NO: 1. The motif may be selected from residues 783-793, 783-792, 783-791, 783-790, 783-789 or 783-788 of SEQ ID NO:1. Also, the motif may be selected from residues 784-793, 785-792 or 786-790 of SEQ ID NO:1.

Alternatively, in aspects of the invention where a catalytically inactive version of Cpf1 is provided, the RuvC domain may comprise a Glu (E) residue, and short motif Glu-lle-Asp (GID).

Alternatively, in aspects of the invention where a catalytically inactive version of Cpf1 is provided, the RuvC domain may comprise a Glu (E) residue, and short motif Gly-lle-Asp (GID).

In aspects of the invention where a catalytically inactive version of Cpf1 is provided, the RuvC domain may comprise a Glu (E) residue, and short motif Glu-lle-Asp (EID).

In aspects of the invention where a catalytically inactive version of Cpf1 is provided, the RuvC domain may comprise a Glu (E) residue, and short motif Ser-lle-Asp (SID).

In aspects of the invention where a catalytically inactive version of Cpf1 is provided, the RuvC domain may comprise the amino acid sequence motif: X⁸IDRGER wherein X⁸ is one of G or S (SEQ ID NO: 7).

In aspects of the invention where a catalytically inactive version of Cpf1 is provided, the RuvC domain may comprise the amino acid sequence motif: DANGAY (SEQ ID NO: 8).

In aspects of the invention where a catalytically inactive version of Cpf1 is provided, the RuvC domain may comprise the amino acid sequence motif: EX⁹LN wherein X⁹ is one of D, Nor E (SEQ ID NO: 9).

In aspects of the invention where a catalytically inactive version of Cpf1 is provided, the RuvC domain may comprise the amino acid sequence motif: EDLN (SEQ ID NO: 10).

A polypeptide or fragment of the invention may be defined both in terms of the reference sequence SEQ ID NO:1 and any percentage variant thereof, in combination with any of the aforementioned amino acid motifs as essential features.

In any aspect of the invention herein, the protein or polypeptide may have an RuvC (nuclease) domain.

In accordance with the invention, the RuvC domain may comprise a short motif GID.

In accordance with the invention, the RuvC domain may comprise a short motif SID.

In accordance with the invention, the RuvC domain may comprise a Glu (E) residue, and short motif GID.

The RuvC domain may comprise a Glu (E) residue, and short motif SID.

Where the RuvC domain comprises a Glu (E) residue, and short motif GID or SID, the D (aspartate) residue of the motif may be a catalytic residue.

The RuvC domain may comprise the amino acid sequence motif X⁸IDRGER (SEQ ID NO: 7) wherein X⁸ is one of G or S. For example, the protein or polypeptide may have an RuvC (nuclease) domain, wherein the RuvC domain comprises the amino acid sequence motif SIDRGER (SEQ ID NO: 11).

Where the RuvC domain comprises an amino acid sequence motif GIDRGER (SEQ ID NO: 12) or SIDRGER (SEQ ID NO: 11), the D (aspartate) residue of the motif may be a catalytic residue.

The protein or polypeptide may have an RuvC (nuclease) domain, wherein the RuvC domain may comprise the amino acid sequence motif DANGAY (SEQ ID NO: 8).

Where the RuvC domain comprises an amino acid sequence motif DANGAY (SEQ ID NO: 8), the D (aspartate) residue of the motif may be a catalytic residue.

The protein or polypeptide may have an RuvC (nuclease) domain, wherein the RuvC domain may comprise the amino acid sequence motif: EX⁹LN wherein X⁹ is one of D, N or E (SEQ ID NO: 9). For example, the protein or polypeptide may have an RuvC (nuclease) domain, wherein the RuvC domain comprises the amino acid sequence motif: EDLN. Where the RuvC domain comprises an amino acid sequence motif EDLN (SEQ ID NO: 10), ENLN (SEQ ID NO: 13) or EELN (SEQ ID NO: 14), the E (glutamate) residue of the motif may be a catalytic residue.

In accordance with the invention, the polypeptide or fragment may have an RuvC (nuclease) domain comprising a Glu (E) residue, and the amino acid sequence motifs SID and DANGAY (SEQ ID NO: 8).

Optionally, the polypeptide or fragment may have an RuvC (nuclease) domain comprising a Glu (E) residue, and the amino acid sequence motifs SID and EDLN (SEQ ID NO: 10).

Optionally, the polypeptide or fragment may have an RuvC (nuclease) domain comprising a Glu (E) residue, and the amino acid sequence motifs SID, DANGAY (SEQ ID NO: 8) and EDLN (SEQ ID NO: 10).

Optionally, the RuvC (nuclease) domain may comprise the amino acid sequence motif: X⁸IDRGER wherein X⁸ is one of G or S, and the amino acid sequence motif DANGAY (SEQ ID NO: 8).

Optionally, the RuvC (nuclease) domain may comprise the amino acid sequence motif: X⁸IDRGER wherein X⁸ is one of G or S (SEQ ID NO: 7), and the amino acid sequence motif: EX⁹LN wherein X: is one of D, Nor E (SEQ ID NO: 9).

Optionally, the RuvC (nuclease) domain may comprise the amino acid sequence motif: X⁸IDRGER wherein X⁸ is one of G or S (SEQ ID NO: 7), and the amino acid sequence motif: EDLN (SEQ ID NO: 10).

Optionally, the RuvC (nuclease) domain may comprise the amino acid sequence motif: X⁸IDRGER wherein X⁸ is one of G or S (SEQ ID NO: 7), and the amino acid sequence motif: DANGAY (SEQ ID NO: 8) and the amino acid sequence motif: EX^{9L}N wherein X⁹ is one of D, Nor E (SEQ ID NO: 9).

Optionally, the RuvC (nuclease) domain may comprise the amino acid sequence motif: X⁸IDRGER wherein X⁸ is one of G or S (SEQ ID NO: 7), and the amino acid sequence motifs DANGAY (SEQ ID NO: 8) and EDLN (SEQ ID NO: 10).

Preferably, the RuvC (nuclease) domain will comprise the amino acid sequence motifs: SIDRGER (SEQ ID NO: 11), DANGAY (SEQ ID NO: 8) and EDLN (SEQ ID NO: 10).

In other aspects, the polypeptide or fragment may have an Arginine-rich motif.

The Arginine-rich motif may comprise the amino acid sequence motif: X¹⁰YX¹¹X¹²X¹³LX¹⁴X¹⁵X¹⁶EX¹⁷X¹⁸X¹⁹X²⁰X²¹ARX²²X²³, wherein X¹⁰ is one of D or N, X¹¹ is one of R, Q or H, X¹² is one of K, E, S or D, X¹³ is one of A, K or L, X¹⁴ is one of D, N or A, X¹⁵ is one of V, N, Q, K or A, X¹⁶ is one of R, K or I, X¹⁷ is one of Y, K or I, X¹⁸ is one of D or E, X¹⁹ is one of N, R or M, X²⁰ is one of K, V, F or D, X²¹ is one of E, A, D or S, X²² is one of R, Q or K and X²³ is one of N, A, S or D (SEQ ID NO: 15).

The Arginine-rich motif may comprise the amino acid sequence motif:

(SEQ ID NO: 16)
DYRKALDVREYDNKEARRN,
(SEQ ID NO: 17)
DYQKKLDNREKERVAARQA,
(SEQ ID NO: 18)
DYREKLNQREIEMKDARQS,
(SEQ ID NO: 19)
DYHSLLDKKEKERFEARQN,
or
(SEQ ID NO: 20)
NYHDKLAAIEKDRDSARKD.

In accordance with the invention, the polypeptide or fragment may have an RuvC (nuclease) domain comprising a Glu (E) residue, and the amino acid sequence motifs Ser-lle-Asp (SID), DANGAY (SEQ ID NO: 8) and EDLN (SEQ ID NO: 10) the amino acid sequence motif. Preferably, the RuvC (nuclease) domain will comprise the amino acid sequence motif: X⁸IDRGER wherein X⁸ is one of G or S (SEQ ID NO: 7), and the amino acid sequence motifs DANGAY (SEQ ID NO: 8) and EDLN (SEQ ID NO: 10). More preferably, the RuvC (nuclease) domain will comprise the amino acid sequence motifs: SIDRGER (SEQ ID NO: 11), DANGAY (SEQ ID NO: 8) and EDLN (SEQ ID NO: 10).

In further embodiments a polypeptide or fragment of any aspect of the invention preferably does not comprise an HNH (nuclease) domain. Additionally or alternatively, in yet a further embodiment, a polypeptide or fragment of any aspect of the invention does not comprise a recognition lobe that is typically present in Cas9. Certain polypeptides or fragments of the invention may have nuclease activity that is provided by a single site in the polypeptide.

Other polypeptides or fragments of the invention may further comprise a zinc finger-domain, although the metal-binding site (typically 4 amino acids, Cys and/or His) is not complete in all Cpf1 variants.

Polypeptides or fragments of the invention may have a nuclease activity which is single strand cleavage, e.g. nickase activity.

Preferably, two subunits of Cpf1 may be used in a dimeric arrangement where nuclease domains of each of the two subunits cleave individual DNA strands. Preferably, such a dimer may be a homodimer where the RuvC-like domains of each of the two subunits cleave individual DNA strands. Alternatively, Cpf1 polypeptides of the invention may be engineered to contain more than one nuclease domain, native or otherwise, which permit cleavage of both DNA strands.

Polypeptide or fragments of the invention preferably have binding affinity for a guide RNA molecule.

In other aspects, a polypeptide or fragment of the invention may have a guide RNA comprising a sequence substantially complementary to a sequence comprised in a target nucleic acid strand.

In further embodiments, a polypeptide or fragment of the invention preferably has binding affinity for a polynucleotide sequence motif in a target nucleic acid strand. This sequence motif is usually known as a protospacer adjacent motif (PAM) sequence. Preferably the nucleotide sequence motif is at least 3 contiguous nucleic acid residues.

The PAM is located on the target (adjacent to protospacer). Typically, the SEED domain of the guide RNA (the region most likely responsible for initial guide/target base pairing) is complementary to the target nucleic acid sequence. Preferably, the SEED part of the guide does not tolerate mismatches.

In order to further improve the polypeptides or fragments of the invention, additional amino acids may be added, preferably by way of a fusion to the N or C terminus. The additional amino acid sequence may have nucleic acid or chromatin modifying, visualising, transcription activating or transcription repressing activity and is preferably translationally fused through expression in natural or artificial protein expression systems, or covalently linked by a chemical synthesis step to the at least one subunit; preferably the at least one functional moiety is fused or linked to at least the region of the N terminus and/or the region of the C terminus.

The additional amino acid sequence having nucleic acid or chromatin modifying, activating, repressing or visualising activity may be a protein; optionally selected from a helicase, a nuclease, a nuclease-helicase, a DNA methyltransferase (e.g. Dam), or DNA demethylase, a histone methyltransferase, a histone demethylase, an acetylase, a deacetylase, a phosphatase, a kinase, a transcription (co-)activator, an RNA polymerase subunit, a transcription repressor, a DNA binding protein, a DNA structuring protein, a marker protein, a reporter protein, a fluorescent protein, a ligand binding protein (e.g. mCherry or a heavy metal binding protein), a signal peptide (e.g. TAT-signal sequence), a subcellular localisation sequence (e.g. nuclear localisation sequence) or an antibody epitope.

When the protein is a nuclease, it may be one selected from a type II restriction endonuclease such as Fokl, or a mutant or an active portion thereof. Preferably, one protein complex of the invention may be fused to the N terminal domain of Fokl and another protein complex of the invention may be fused to the C terminal domain of Fokl. These two protein complexes may then be used together (in a dimeric configuration) to achieve an advantageous locus specific double stranded cut in a nucleic acid, whereby the location of the cut in the genetic material is at the design and choice of the user, as guided by the RNA component (defined and described below) and due to presence of a so-called “protospacer adjacent motif” (PAM) sequence in the target nucleic acid strand (also described in more detail below).

In a preferred embodiment, a protein or polypeptide of the invention has an additional amino acid sequence which is a modified restriction endonuclease, e.g. Fokl. The modification is preferably in the catalytic domain. In preferred embodiments, the modified Fokl is KKR Sharkey or ELD Sharkey, which is fused to the Cpf1 protein. In a preferred application of these complexes of the invention, two of these complexes (KKR Sharkey and ELD Sharkey) may be together in combination. A heterodimer pair of protein complexes employing differently modified Fokl has particular advantage in targeted double stranded cutting of nucleic acid. If homodimers are used then it is possible that there is more cleavage at non-target sites due to non-specific activity. A heterodimer approach advantageously increases the fidelity of the cleavage in a sample of material.

Advantageously the above modifications can permit a user to select in a predetermined manner a precise genetic locus which is desired to be cleaved, tagged or otherwise altered in some way, e.g. methylation, using any of the nucleic acid or chromatin modifying, visualising, transcription activating or transcription repressing entities defined herein. The other component part of the system is an RNA molecule which acts as a guide for directing the complexes of the invention to the correct locus on DNA or RNA intending to be modified, cut or tagged.

In further embodiments, a polypeptide or fragment of the invention is preferably bound to a guide RNA and to a target nucleic acid. In this form a complex is formed which provides targeted DNA strand nuclease activity, wherein a desired target locus is cleaved.

In another aspect the present invention provides a polynucleotide comprising a polynucleotide sequence encoding a polypeptide or fragment of the invention as hereinbefore defined.

In further aspect, the present invention provides an expression vector comprising a polynucleotide as aforementioned.

The invention also provides an expression vector as defined above, further comprising a nucleotide sequence encoding a guide RNA which has substantial complementarity to a desired sequence in the target nucleic acid strand. Guide RNA in the native state is a single RNA consisting of a crRNA.

The invention further provides an expression vector of the invention which is preferably a viral vector, e.g. Adenovirus, or Adena-associated Virus (AAV).

In other aspects, the invention provides a host cell transformed to express a polypeptide or fragment of the invention as hereinbefore described.

Typically, the expression vector DNA can be delivered to the host cell by transformation, electroporation or virus (AAV). Also, RNA can be delivered into a host cell by injection or electroporation. Proteins can be delivered to cells via electroporation, peptide (HIV) tags. In another aspect the present invention provides a host cell as hereinbefore described, additionally transformed to contain a guide RNA comprising a sequence substantially complementary to a sequence comprised in a target nucleic acid strand in the host cell.

The invention includes any host cell transformed with an expression vector as hereinbefore described.

The invention also provides a method of cleaving a target nucleic acid strand at a specific locus, comprising exposing the target nucleic acid to a polypeptide or fragment of the invention, and with a guide RNA molecule which comprises a sequence substantially complementary to a sequence comprised in the target nucleic acid strand.

The invention further provides a method of cleaving a target nucleic acid strand at a specific locus in the genome of a cell of an organism, comprising transforming the cell with an expression vector of the invention as described herein, and transforming the cell with a vector which expresses a guide RNA comprising a sequence substantially complementary to a sequence comprised in a target nucleic acid strand.

In further aspect, the invention provides a method of cleaving a target nucleic acid strand at a specific locus in the genome of a cell of an organism, comprising transforming the cell with an expression vector of the invention as described herein.

In another aspect the present invention provides a method of non-homologous end joining gene editing comprising (a) transforming the cell with an expression vector of the invention, and transforming the cell with a vector which expresses a guide RNA comprising a sequence substantially complementary to a sequence comprised in a target nucleic acid strand; or (b) transforming the cell with an expression vector of the invention. In these aspects of the invention the polypeptides of the invention are modified or used to cause double stranded breaks.

In a further aspect the invention provides a method of homologous end joining gene editing comprising (a) transforming the cell with an expression vector of the invention, and transforming the cell with a vector which expresses a guide RNA comprising a sequence substantially complementary to a sequence comprised in a target nucleic acid strand; or (b) transforming the cell with an expression vector of the invention; so as to create a double strand break at a desired locus in the genetic material, and exposing the genetic material to a polynucleotide sequence which has end regions complementary to the broken end regions of the genetic material.

### DETAILED DESCRIPTION

The protein of amino acid sequence SEQ ID NO: 1 is a large protein (about 1300 amino acids) that contains an RuvC-like nuclease domain homologous to the respective domains of Cas9 and transposable element ORF-B, along with an arginine-rich region similar to that in Cas9 and a Zinc Finger (absent in Cas9 but shared with ORF-8), but lacks the HNH nuclease domain that is present in all Cas9 proteins.

The invention will now be described in detail with reference to the examples and to the drawings in which:

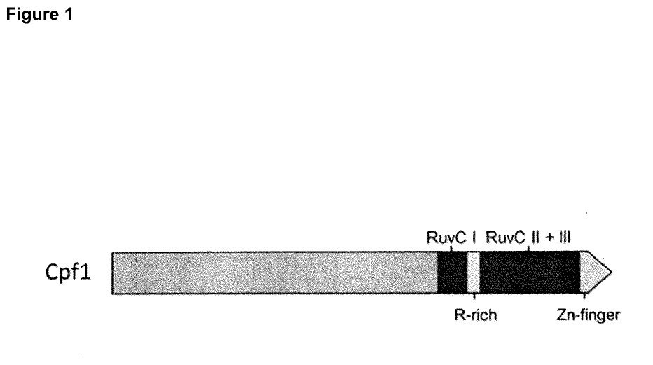

FIG. 1 shows the domain structure of the novel CRISPR-Cas nuclease, Cpf1. Three RuvC nuclease domains, a Zinc-finger and an arginine-rich domain that allows for interaction with RNA guide and DNA target are shown.

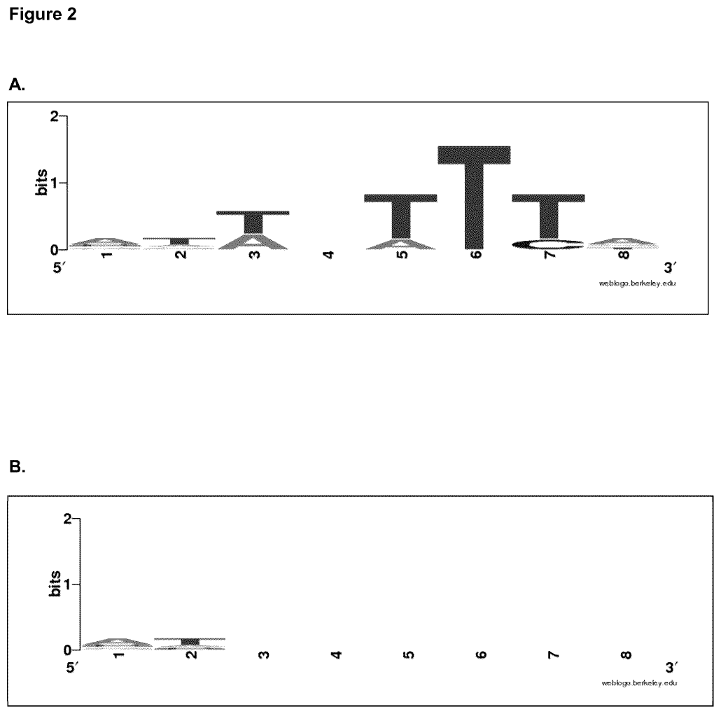

FIG. 2 shows the results of an in silico analysis of conserved Protospacer Adjacent Motif (PAM). Panel A shows a Weblogo based on 5′ flanks of protospacers depicted in Table 1. Panel 8 shows a Weblogo based on 3′ flanks of protospacers depicted in Table 1.

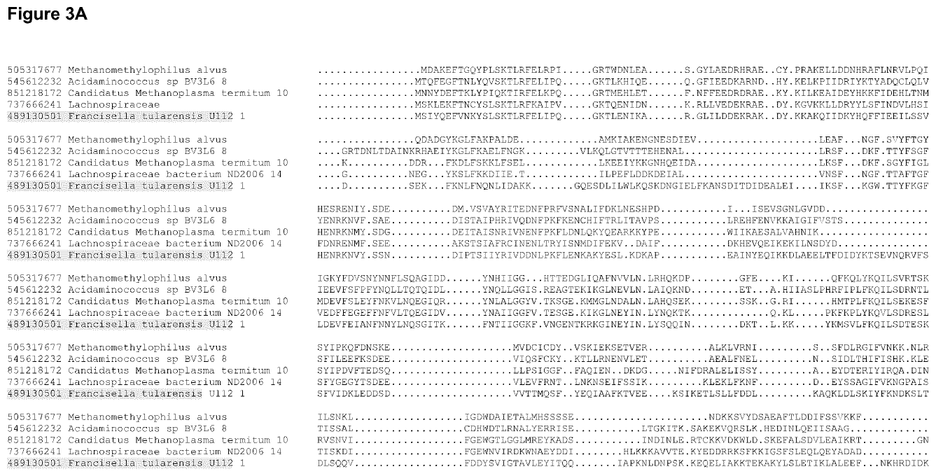

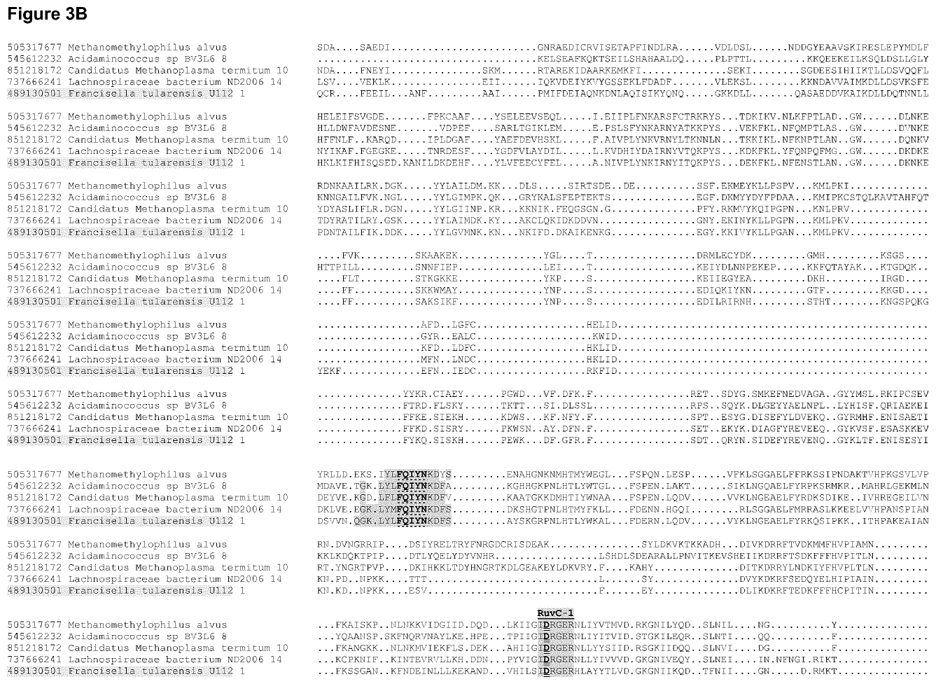

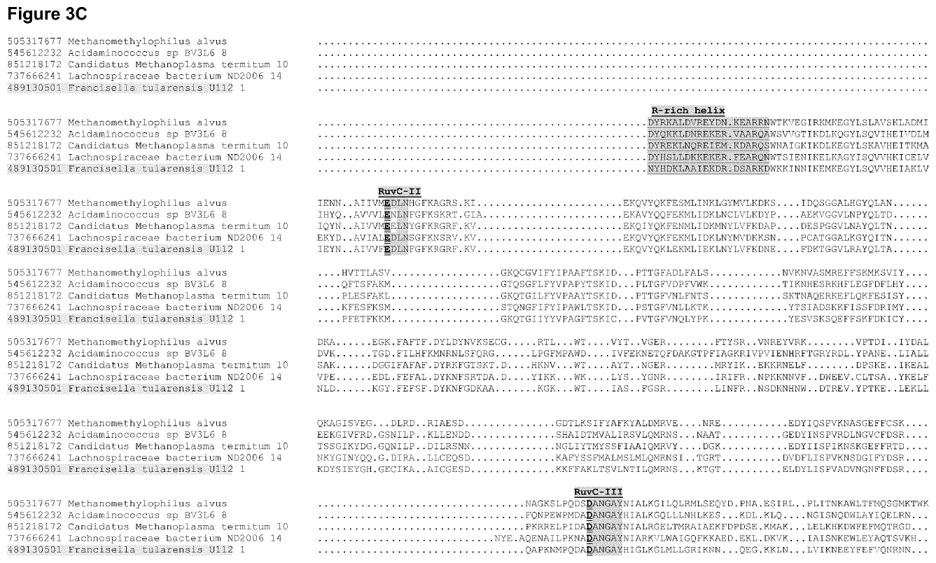

FIGS. 3A-3C show the results of a multiple alignment of the Cpf1 protein family (SEQ ID NOS 32, 33, 36, 34 and 35, respectively, in order of appearance). Each sequence is labelled with GenBank Identifier (GI) number and systematic name of an organism. Predicted secondary structure (SS) is shown by shading. Active site residues of RuvC-like domain(s) are shown as bold and double underlined. Potential bridge helix is shown by shading and with single underline. The amino acid sequence FQIYN (SEQ ID NO: 2) is also indicated in bold, by shading and dotted underline.

### EXAMPLE 1—NOVEL NUCLEASES FOR GENE EDITING

Specific examples are (1) CRISPR-associated Cpf1 from the marine bacterium Francisella novicida (Fn-Cpf1), and (2) CRISPR-associated Cpf1 from the archaean Methanomethylophylus alvus strain Mx1201 (Mai-Cpf1) that resides in the human gut.

Without the inventors wishing to be bound by any particular theory, Cpf1 recognises the crRNA in a sequence-specific manner, after which cleavage occurs of the double stranded RNA segment, and eventually formation of an effector complex consisting of Cpf1 and a single crRNA guide. Cpf1 may operate as a dimer, with the RuvC-like domains of each of the two subunits cleaving individual DNA strands. Alternatively, Cpf1 may contain more than one nuclease domain which permits cleavage of both DNA strands. Alternatively, one or more RuvC domains of Cpf1 may exhibit unusual flexibility that allows for cleavage of both strands.

The following examples were performed in parallel for the bacterial Fno-Cpf1 and archaeal Mai-Cpf1 protein variants:

Cloning is carried out of the entire CRISPR locus, including cas operon (cpf1-cas4-cas1-cas2), leader region, CRISPR array, and flanking regions (approximately 10 kb) in low-copy vector (e.g. pACYC184) in an E. coli K12 strain; no details are known about the maturation of the guide, which may be similar to that of Cas9 (tracrRNA/RNaselll), or may be similar to that of Cascade (Cash-like ribonuclease, although that is not part of cpf1 operons), or may be unique. Further detailed materials and methods are provided in Sapranauskas et al., 2011, Nucleic Acids Res. 39:9275-9282.

Standard procedures were used to optimize chances for functional protein production of the selected Cpf1 proteins in E. coli: (i) by performing codon harmonization design to adjust cpf1 nucleotide sequences (see Angov et al., 2008, PLoS One 3, e2189); (ii) by including N-terminal or C-terminal strepII tag, that will allow for affinity purification; (iii) by cloning synthetic gene in T7 expression vector (e.g. pET24d) and transform plasmid to non-production strain of E. coli (e.g. JM109, lacking T7 RNA polymerase gene), (iv) transferring plasmid via second transformation to production strain of E. coli (e.g., BL21(DE3), containing T7 RNA polymerase gene under control of rhamnose promoter, that allows for accurate tuning of expression, (v) varying expression conditions (medium, inducer concentration, induction time), (vi) using optimal conditions for liter-scale cultivation, after which cells are harvested and mechanically disrupted to obtain cell-free extract (small volumes by sonication; large volumes by French Press), (vii) separating membrane and soluble fractions, and perform affinity purification using streptactin resin, (viii) testing relevant fractions by SDS-PAGE, and storing the pure protein for subsequent analyses.

As well as the above, additionally, the predicted crRNA gene is sequenced, or a single-guide RNA (sgRNA) gene is made, e.g. by adding 4 nucleotide synthetic loops (Jinek et al., 2012, Science 337: 816-821); RNA genes residing either on the same plasmid as cpf1 gene, or on a separate plasmid.

Additionally, a catalytically inactive Cpf1 mutant is made (RuvC active site contains conserved glutamate (E) as well as GID motif).

Additionally, a catalytically inactive Cpf1 mutant is made (RuvC active site contains conserved glutamate (E) as well as SID motif).

Also, N-terminal or C-terminal fusions are made of the Cpf1 mutant with Fokl nuclease domain with differently connecting linkers (as described for Cas9; see Guilinger et al., 2014, Nat. Biotechnol. 32: 577-82).

### EXAMPLE 2—BIOCHEMICAL CHARACTERIZATION OF CPF1 NUCLEASES

These experiments characterize guide surveillance and target cleavage. The CRISPR system is an adaptive immunity system in bacteria and archaea. The CRISPR arrays consist of identical repeats (e.g. 30 bp) and variable spacers (e.g. 35 bp). The adaptive nature of the CRISPR system relies on regular acquisition of new spacers, often corresponding to fragments (protospacers) derived from viruses. Acquisition generally depends on the selection of a protospacer based on the presence of a protospacer adjacent motif (PAM). The presence of this motif is crucial for the eventual interference by the CRISPR-associated effector complex (e.g. Cas9) with its crRNA guide. The PAM motif allows for self versus non-self discrimination: the potential target sequences (i.e. complementary to the crRNA guide sequence) reside both on the host's genome (the self CRISPR array) as well as on the invader's genome (the non-self protospacer); the presence of the protospacer in the invader DNA triggers the effector complex to bind it in a step-wise manner; when perfect base pairing occurs between the sequence of the protospacer immediately adjacent to the PAM (the so-called seed sequence), then base pairing as a zipper, eventually leading to a state of Cas9 to catalyse cleavage of the target DNA strands (see Jinek et al., 2012, Science 337: 816-821; also Gasiunas et al., 2012, PNAS 109: E2579-E2586).

In silico analysis of the Cpf1-associated PAM by BLAST analysis of the CRISPR spacers of the cpf1-loci. BLAST analysis of some spacers shows several homologous sequences (90-100% identity), (Table 1). The most promising hits concern identical sequences of virus genes in general, and genes of prophages in particular. Prophages are derived from lysogenic viruses, the genomes of which have integrated in the genome of bacteria. As is the case with eukaryotic viruses, the host range of prokaryotic viruses is often rather limited; hence, when the matching prophage is found in a bacterium that is closely related to the bacterium that has the corresponding spacer sequence in its CRISPR array, this gives some confidence that it is a real hit. In other words, it may well be that the prophage resembles a virus that has attempted to infected the CRISPR-containing bacterium, but the invasion has resulted in spacer acquisition and virus immunity of the latter bacterium.

TABLE 1
BLAST results with FnU112 cpf1-associated CRISPR spacers as
query sequences. The nucleotide sequence of both spacer (top) and protospacer are shaded; the
5′ and 3′ flanks of the protospacers are unshaded; Tool: CRISPR Target
(bioanalysis.otago.ac.nz/CRISPRTarget/). Query: Entire CRISPR array from Francisella
novicida sub species. Target database: Genbank-NT. Gap open -10, Extend -2; Nucleotide
match 1, mismatch -1; E-value 1; Word size 7; Cutoff score 20; 3′ end flanking protospacer 8
bp; 5′ end flanking protospacer 8 bp.

| | Host of | | | |
|---|---|---|---|---|
| | prophage, | | | |
| Fn sub | target gene | | Alignment of Fn sub species | |
| species | accession | | spacer with protospacer (plus | SEQ ID |
| Spacer# | number | | 8 nt flanks on both sides) | NO: |
| Francisella | Francisella | spacer | 5′ AGATTAAAAGGTAATTCTATCTTGTTGAG | 21 |
| novicida | novicida | protospacer | \|\|\|\|\|\|\|\|\|\|\|\|\|\|\|\|\|\|\|\|o\|\|\|\|\|\|\|\| | |
| U112 #1 | 3523, hypo | | 5′ ATAATTTAAGATTAAAAGGTAATTCTATTTTG | 22 |
| | prot | | TTGAGATCTGAGC | |
| | AEE26301.1 | | | |
| Francisella | Francisella | Spacer | 5′ TAGCGATTTATGAAGGTCATTTTTTT | 23 |
| novicida | novicida | protospacer | \|\|\|\|\|\|\|\|\|\|\|\|\|\|\|\|\|\|\|\| | |
| U112 #2 | 3523, | | 5′ CTAAATTATAGCGATTTATGAAGGTCATTTTT | 24 |
| | intergenic | | TTAAAAAGTT | |
| | sequence in | | | |
| | prophage | | | |
| Francisella | Francisella | Spacer | 5′ ATGGATTATTACTTAACTGGAGTGTTTAC | 25 |
| novicida | novicida | protospacer | \|\|\|\|\|\|\|\|\|\|\|\|\|\|\|\|\|\|\|\|o\|\|\|\|\|\|\|o\|\|\| | |
| Fx1 #1 | 3523, hypo | | 5′ AATGTTCAATGGATTATTACTTAATTGGAGTG | 26 |
| | prot | | TCTACGTCGATGG | |
| | AEE26295.1, | | | |
| | “phage major | | | |
| | tail tube | | | |
| | protein” | | | |
| Francisella | Francisella | Spacer | 5′ GCCACAAATACTACAAAAAATAACTTAA | 27 |
| novicida | novicida | protospacer | \|\|oo\|\|\|\|\|\|\|\|\|\|\|\|\|\|\|\|\|\| | |
| FTG #1 | 3523, hypo | | 5′ATTTTTTGGCTCCAAATACTACAAAAAATAAC | 28 |
| | prot | | TTAAACTTTGAA | |
| | YP_ | | | |
| | 005824059.1 | | | |
| Francisella | Francisella | Spacer | 5′ ATTGTCAAAACATAAGCAGCTGCTTCAAATAT | 29 |
| novicida | novicida3523, | protospacer | \|o\|\|\|o\|oo\|\|\|\|\|\|\|\|\|\|o\|\|\|\|\|\|\|\|\|\|\| | |
| GA99- | hypoprot | | 5′GGTCTTTTACTGTTATTACATAAGCAGCCGCT | 30 |
| 3549 #1 | FN3523_1009, | | TCAAATATCTTAGCAA | |
| | “baseplate_J” | | | |

Analysis of the sequences flanking the protospacers in the prophage genes resulted in a T-rich conserved motif; interestingly, this motif does not reside downstream the protospacer (as in the Cas9 system), but rather upstream. Though not wishing to be bound by particular theory, the inventors find that Cpf1 of the invention requires a PAM-like motif (3-4 nucleotides) for binding a target DNA molecule that is complementary to the guide, has a seed sequence (8-10 nucleotides) in which no mismatches are allowed, and has a single nuclease site that allows for nicking of the base paired target DNA strand.

PAM motifs of Cpf1 and variants of the invention were also characterized using the approach of Jiang et al., 2013, Nat. Biotechnol. 31: 233-239). Two derivatives of E. coli BL21(DE3) were used, initially transformed either with a target-plasmid or with a non-target plasmid; two variant target plasmids used have a similar part (GFP marker, KmR marker, origin of replication) and a variable part with target sequence (protospacer) with an associated degenerate PAM (5-8 variable nucleotides) either upstream or downstream of the protospacer); next, this strain was transformed with a Cpf1-expression plasmid (includes design-CRISPR with single-guide RNA (sgRNA, CmR-marker); screening for transformants was on plates with chloramphenicol (Cm) (not kanamycin (Km)), and screening for non-fluorescent colonies, indicating loss-of-target-plasmid. As the plasmids with the correct PAMs will be lost, DNA Deep Seq was performed of appropriate PCR products of the entire pool of target plasmid, before and after transformation. The differences reveal the PAM (Bikard et al., 2013, Nucleic Acids Res. 41: 7429-7437).

PAM signatures were confirmed by in vitro characterization of cleavage activity of BsCas9/sgRNA; assays reveal optimal conditions (temperature, buffer/pH, salt, metals).

Presence of a seed sequence in the PAM was established according to methods described by Jinek et al., 2012, Science 337: 816-821.

### EXAMPLE 3—BACTERIAL ENGINEERING

Performing of high-throughput engineering of bacterial genome with nuclease variants. Without wishing to be bound by particular theory, the inventors expect that Cpf1/guide complexes of the invention allow for specific targeting of genomic DNA. Multiplex targeting can be established by using a design CRISPR together with a matching crRNA.

The experiments provide application of Cpf1 and variants of the invention. Cas9 is tested in parallel as a reference.

Gene knock-in/knock-out (insertion/disruption of any sequence) is performed. The host strain E. coli K12 (LacZ+, GFP−) was engineered as follows: the gene encoding a variant of the Green Fluorescent Protein (GFPuv) is inserted in the lacZ gene, resulting in a clear phenotype (LacZ−, GFP+). The cpf1 gene was introduced on a plasmid (or derivatives of those plasmids), together with a fragment that allows for homologous recombination of the target sequence. A target (protospacer) sequence was selected, with an appropriate adjacently located PAM sequence; a corresponding guide designed, consisting of the crRNA (with spacer complementary to target protospacer) and the crRNA gene (as adapted from the method described for Cas9 by Jiang et al. (2013a) RNA-guided editing of bacterial genomes using CRISPR-Cas systems. Nat. Biotechnol. 31: 233-239).

Gene expression silencing (using catalytically inactivated Cas9, was as described: dCas9 derivative of Spy-Cas9; (Bikard et al., 2013, Nucleic Acids Res. 41: 7429-7437; Qi et al., 2013, Cell 152: 1173-1183);) by binding at promoter (RNA polymerase binding site) of target gene, or of target genes using a multiplex approach (using a design CRISPR).

Gene expression activation; as above (silencing); binding upstream binding site of RNA polymerase, with Cas9 fused to activation domain (as has been described for Spy-Cas9) (Bikard et al., 2013, Nucleic Acids Res. 41: 7429-7437).

Fusion of inactivated Cpf1 and the Fokl nuclease domain (described in Example 1 were compared with an active Cpf1 in different experimental set-ups. This required two simultaneous interactions of guides and targets, that results in a major improvement of cleavage at the desired site.

### EXAMPLE 4—HUMAN STEM CELL ENGINEERING

Targeted editing of disease-causing genetic mutations would be an elegant and effective treatment for genetic disease. Recently discovered gene editing systems such as Cas9, allow the specific targeting of disease-causing mutations in the genome, and can be used to functionally repair or permanently disable mutated genes. The efficiency of gene editing systems has been demonstrated in a laboratory setting, and are now routinely used in genome editing of a wide variety of cell types from many different species, including human. However, despite the success of these systems in the research setting, clinical application of gene editing systems is hampered by the lack of a suitable delivery system to introduce gene-editing technologies into patient cells in a safe, transient and efficient manner. Several labs are working on the development of recombinant viral vectors which can be used to deliver gene editing systems into patient cells, but prolonged expression of for example CRISPR/Cas9 from such vectors will increase the likelihood of off-target effects and is therefore not ideal. Intracellular delivery of recombinant gene editing protein and synthetic CRISPR RNA would be an effective, non-integrating and transient method for the application of gene editing technology in patient cells.

Recently a novel method has been developed that allows the transduction of native proteins into virtually any cell type (D'Astolfo et al., 2015, Cell, 161: 674-690). This technology, termed iTOP, for induced Transduction by Osmocytosis and Propanebetaine, is based on a combination of small molecule compounds, which trigger the uptake and intracellular release of native protein. iTOP is highly efficient, routinely achieving transduction efficiencies of >90% of cells, and works on a wide variety of primary cell types. It has been demonstrated that iTOP-mediated transduction of recombinant Cas9 protein and in vitro transcribed sgRNA allows for highly efficient gene editing in difficult-to-transfect cell types including human stem cells. Upon iTOP-CRISPR/Cas9 transduction, >70% bi-allelic gene targeting has been reported in human ES cells without the need for drug-selection of transduced cells.

Key advantages of iTOP over existing technologies are: (i) the ability to transduce primary (stem) cells with native protein at very high efficiency, (ii) the non-integrating, transient nature of protein mediated gene editing, ensuring safety and minimizing off-target effects, and (iii) the tight control of dosage and timing of the delivered protein. We have demonstrated that iTOP-CRISPR/Cas9 is an effective tool to modify a large variety of primary (patient) cell types. However, due to size and protein solubility issues, production of recombinant Cas9 is hampering broad-scale (clinical) adoption of this system. Cpf1 could solve these problems and pave the way for the development of novel therapies to treat genetic disease.

The iTOP technology will be used to allow efficient intracellular delivery of Cpf1 into human stem cells. The advantage of iTOP is its highly flexible approach. First, NaCl-mediated hypertonicity induces intracellular uptake op protein via a process called macropinocytosis (D'Astolfo op. cit.)). Second, a propanebetaine transduction compound (NDSB-201 or gamma-aminobutyric acid (GABA) or others triggers the intracellular release of protein from the macropinosome vesicles. In addition to these compounds, osmoprotectants such as glycerol and glycine are added to help cells to cope with the NaCl-induced hypertonic stress. By varying the concentration of NaCl, the concentration and type of transduction compound and/or the concentration and type of osmoprotectants, the iTOP system can be adapted and optimised to meet the specific requirements of the cargo protein and/or the target cells. iTOP parameters were optimized to allow efficient gene editing of human embryonic stem cells (hESCs), targeting the endogenous WDR85 gene by Cpf1 (equipped with an N- or C-terminal nuclear localization signal (NLS)), as recently shown for Cas9.

In the following sequence listing, the amino acid residues Glu Xaa Asp (single underlined) are the GID motif of an RuvC domain. Therefore in the SEQ ID NO: 1, the Xaa residue may be I.

The amino acid residues lie Asp Arg Gly Glu Arg (double underlined) include the IDR residues of an RuvC domain.

The amino acid residues Phe Glu Asp (triple underlined) include the E residue making up part of the active site residues of an RuvC domain.

### EXAMPLE 5 MULTIPLE ALIGNMENT OF CPF1 PROTEINS

FIGS. 3A-3C show the results of an Multiple alignment of Cpf1 proteins. The alignment was built using MUSCLE program and modified manually on the basis of local PSI-BLAST pairwise alignments and HHpred output. Each sequence is labelled with GenBank Identifier (GI) number and systematic name of an organism. Five sequences analysis in this work are marked by the respective numbers. Secondary structure (SS) was predicted by Jpred and is shown is shown by shading. CONSENSUS was calculated for each alignment column by scaling the sum-of-pairs score within the column between those of a homogeneous column (the same residue in all aligned sequences) and a random column with homogeneity cutoff 0.8. Active site residues of RuvC-like domain(s) are shown as bold and double underlined. Potential bridge helix is shown by shading and with single underline. The amino acid sequence FQIYN (SEQ ID NO: 2) is also indicated in bold, by shading and dotted underline.

SEQUENCE LISTING

| | |
|---|---|
| <110> | wageningen universiteit |
| <120> | cpf1 Nuclease |
| <130> | RAW/P223284GB |
| <160> | 1 |
| <170> | Patentin version 3.5 |
| <210> | 1 |
| <211> | 1304 |
| <212> | PRT |
| <213> | Artificial sequence |
| <220> | |
| <223> | Cpf1 |
| <220> | |
| <221> | misc_feature |
| <222> | (439)..(439) |
| <223> | Xaa can be any naturally occurring amino acid |
| <220> | |
| <221> | misc_feature |
| <222> | (504)..(504) |
| <223> | Xaa can be any naturally occurring amino acid |
| <220> | |
| <221> | misc_feature |
| <222> | (521)..(521) |
| <223> | Xaa can be any naturally occurring amino acid |
| <220> | |
| <221> | misc_feature |
| <222> | (539)..(539) |
| <223> | Xaa can be any naturally occurring amino acid |
| <220> | |
| <221> | misc_feature |
| <222> | (800)..(800) |
| <223> | Xaa can be any naturally occurring amino acid |
| <400> | 1 |

```
Met ser Ile Tyr Gln Glu Phe Val Asn Lys Tyr Ser Leu Ser Lys Thr
1 5 10 15
Leu Arg Phe Glu Leu Ile Pro Gln Gly Lys Thr Leu Glu Asn Ile Lys
                        20 25 30
Ala Arg Gly Leu Ile Leu Asp Asp Glu Lys Arg Ala Lys Asp Tyr Lys
                35 40 45
Lys Ala Lys Gln Ile Ile Asp Lys Tyr His Gln Phe Phe Ile Glu Glu
        50 55 60
Ile Leu Ser Ser Val Cys Ile Ser Glu Asp Leu Leu Gln Asn Tyr Ser
65 70 75 80
Asp Val Tyr Phe Lys Leu Lys Lys Ser Asp Asp Asp Asn Leu Gln Lys
                                85 90 95
Asp Phe Lys Ser Ala Lys Asp Thr Ile Lys Lys Gln Ile Ser Glu Tyr
                        100 105 110
Ile Lys Asp Ser Glu Lys Phe Lys Asn Leu Phe Asn Gln Asn Leu Ile
                115 120 125
Asp Ala Lys Lys Gly Gln Glu Ser Asp Leu Ile Leu Trp Leu Lys Gln
        130 135 140
Ser Lys Asp Asn Gly Ile Glu Leu Phe Lys Ala Asn Ser Asp Ile Thr
145 150 155 160
Asp Ile Asp Glu Ala Leu Glu Ile Ile Lys Ser Phe Lys Gly Trp Thr
                                165 170 175
Thr Tyr Phe Lys Gly Phe His Glu Asn Arg Lys Asn val Tyr ser ser
                        180 185 190
Asp Asp Ile Pro Thr ser Ile Ile Tyr Arg Ile val Asp Asp Asn Leu
                195 200 205
Pro Lys Phe Leu Glu Asn Lys Ala Lys Tyr Glu Ser Leu Lys Asp Lys
        210 215 220
Ala Pro Glu Ala Ile Asn Tyr Glu Gln Ile Lys Lys Asp Leu Ala Glu
225 230 235 240
Glu Leu Thr Phe Asp Ile Asp Tyr Lys Thr ser Glu Val Asn Gln Arg
                                245 250 255
Val Phe Ser Leu Asp Glu Val Phe Glu Ile Ala Asn Phe Asn Asn Tyr
                        260 265 270
Leu Asn Gln ser Gly Ile Thr Lys Phe Asn Thr Ile Ile Gly Gly Lys
                275 280 285
Phe val Asn Gly Glu Asn Thr Lys Arg Lys Gly Ile Asn Glu Tyr Ile
        290 295 300
Asn Leu Tyr Ser Gln Gln Ile Asn Asp Lys Thr Leu Lys Lys Tyr Lys
305 310 315 320
Met Ser Val Leu Phe Lys Gln Ile Leu Ser Asp Thr Glu Ser Lys Ser
                                325 330 335
Phe val Ile Asp Lys Leu Glu Asp Asp Ser Asp Val Val Thr Thr Met
                        340 345 350
Gln ser Phe Tyr Glu Gln Ile Ala Ala Phe Lys Thr Val Glu Glu Lys
                355 360 365
Ser Ile Lys Glu Thr Leu Ser Leu Leu Phe Asp Asp Leu Lys Ala Gln
        370 375 380
Lys Leu Asp Leu Ser Lys Ile Tyr Phe Lys Asn Asp Lys Ser Leu Thr
385 390 395 400
Asp Leu ser Gln Gln val Phe Asp Asp Tyr ser val Ile Gly Thr Ala
                                405 410 415
Val Leu Glu Tyr Ile Thr Gln Gln Val Ala Pro Lys Asn Leu Asp Asn
                        420 425 430
Pro ser Lys Lys Glu Gln Xaa Leu Ile Ala Lys Lys Thr Glu Lys Ala
                435 440 445
Lys Tyr Leu Ser Leu Glu Thr Ile Lys Leu Ala Leu Glu Glu Phe Asn
        450 455 460
Lys His Arg Asp Ile Asp Lys Gln cys Arg Phe Glu Glu Ile Leu Ala
465 470 475 480
Asn Phe Ala Ala Ile Pro Met Ile Phe Asp Glu Ile Ala Gln Asn Lys
                                485 490 495
Asp Asn Leu Ala Gln Ile Ser Xaa Lys Tyr Gln Asn Gln Gly Lys Lys
                        500 505 510
Asp Leu Leu Gln Ala ser Ala Glu xaa Asp val Lys Ala Ile Lys Asp
                515 520 525
Leu Leu Asp Gln Thr Asn Asn Leu Leu His Xaa Leu Lys Ile Phe His
        530 535 540
Ile ser Gln Ser Glu Asp Lys Ala Asn Ile Leu Asp Lys Asp Glu His
545 550 555 560
Phe Tyr Leu Val Phe Glu Glu Cys Tyr Phe Glu Leu Ala Asn Ile Val
                                565 570 575
Pro Leu Tyr Asn Lys Ile Arg Asn Tyr Ile Thr Gln Lys Pro Tyr Ser
                        580 585 590
Asp Glu Lys Phe Lys Leu Asn Phe Glu Asn Ser Thr Leu Ala Asn Gly
                595 600 605
Trp Asp Lys Asn Lys Glu Pro Asp Asn Thr Ala Ile Leu Phe Ile Lys
        610 615 620
Asp Asp Lys Tyr Tyr Leu Gly val Met Asn Lys Lys Asn Asn Lys Ile
625 630 635 640
Phe Asp Asp Lys Ala Ile Lys Glu ASn Lys Gly Glu Gly Tyr Lys Lys
                                645 650 655
Ile val Tyr Lys Leu Leu Pro Gly Ala Asn Lys Met Leu Pro Lys val
                        660 665 670
Phe Phe Ser Ala Lys Ser Ile Lys Phe Tyr Asn Pro Ser Glu Asp Ile
                675 680 685
Leu Arg Ile Arg Asn His Ser Thr His Thr Lys Asn Gly Asn Pro Gln
        690 695 700
Lys Gly Tyr Glu Lys Phe Glu Phe Asn Ile Glu Asp cys Arg Lys Phe
705 710 715 720
Ile Asp Phe Tyr Lys Glu Ser Ile Ser Lys His Pro Glu Trp Lys Asp
                                725 730 735
Phe Gly Phe Arg Phe Ser Asp Thr Gln Arg Tyr Asn Ser Ile Asp Glu
                        740 745 750
Phe Tyr Arg Glu val Glu Asn Gln Gly Tyr Lys Leu Thr Phe Glu Asn
                755 760 765
Ile Ser Glu Ser Tyr Ile Asp Ser Val Val Asn Gln Gly Lys Leu Tyr
        770 775 780
Leu Phe Gln Ile Tyr Asn Lys Asp Phe Ser Ala Tyr Ser Lys Gly Xaa
785 790 795 800
Pro Asn Leu His Thr Leu Tyr Trp Lys Ala Leu Phe Asp Glu Arg Asn
                                805 810 815
Leu Gln Asp Val Val Tyr Lys Leu Asn Gly Glu Ala Glu Leu Phe Tyr
                        820 825 830
Arg Lys Gln Ser Ile Pro Lys Lys Ile Thr His Pro Ala Lys Glu Ala
                835 840 845
Ile Ala Asn Lys Asn Lys Asp Asn Pro Lys Lys Glu Ser Val Phe Glu
        850 855 860
Tyr Asp Leu Ile Lys Asp Lys Arg Phe Thr Glu Asp Lys Phe Phe Phe
865 870 875 880
His Cys Pro Ile Thr Ile Asn Phe Lys Ser Ser Gly Ala Asn Lys Phe
                                885 890 895
Asn Asp Glu Ile Asn Leu Leu Leu Lys Glu Lys Ala Asn Asp Val His
                        900 905 910
Ile Leu Ser Ile Asp Arg Gly Glu Arg His Leu Ala Tyr Tyr Thr Leu
                915 920 925
Val Asp Gly Lys Gly Asn Ile Ile Lys Gln Asp Thr Phe Asn Ile Ile
        930 935 940
Gly Asn Asp Arg Met Lys Thr Asn Tyr His Asp Lys Leu Ala Ala Ile
945 950 955 960
Glu Lys Asp Arg Asp Ser Ala Arg Lys Asp Trp Lys Lys Ile Asn Asn
                                965 970 975
Ile Lys Glu Met Lys Glu Gly Tyr Leu Ser Gln Val Val His Glu Ile
                        980 985 990
Ala Lys Leu val Ile Glu Tyr Asn Ala Ile Val Val Phe Glu Asp Leu
                995 1000 1005
Asn Phe Gly Phe Lys Arg Gly Arg Phe Lys Val Glu Lys Gln Val
        1010 1015 1020
Tyr Gln Lys Leu Glu Lys Met Leu Ile Glu Lys Leu Asn Tyr Leu
        1025 1030 1035
Val Phe Lys Asp Asn Glu Phe Asp Lys Thr Gly Gly Val Leu Arg
        1040 1045 1050
Ala Tyr Gln Leu Thr Ala Pro Phe Glu Thr Phe Lys Lys Met Gly
        1055 1060 1065
Lys Gln Thr Gly Ile Ile Tyr Tyr Val Pro Ala Gly Phe Thr Ser
        1070 1075 1080
Lys Ile Cys Pro Val Thr Gly Phe Val Asn Gln Leu Tyr Pro Lys
        1085 1090 1095
Tyr Glu Ser Val Ser Lys Ser Gln Glu Phe Phe Ser Lys Phe Asp
        1100 1105 1110
Lys Ile Cys Tyr Asn Leu Asp Lys Gly Tyr Phe Glu Phe Ser Phe
        1115 1120 1125
Asp Tyr Lys Asn Phe Gly Asp Lys Ala Ala Lys Gly Lys Trp Thr
        1130 1135 1140
Ile Ala Ser Phe Gly Ser Arg Leu Ile Asn Phe Arg Asn Ser Asp
        1145 1150 1155
Lys Asn His Asn Trp Asp Thr Arg Glu Val Tyr Pro Thr Lys Glu
        1160 1165 1170
Leu Glu Lys Leu Leu Lys Asp Tyr Ser Ile Glu Tyr Gly His Gly
        1175 1180 1185
Glu Cys Ile Lys Ala Ala Ile Cys Gly Glu Ser Asp Lys Lys Phe
        1190 1195 1200
Phe Ala Lys Leu Thr Ser Val Leu Asn Thr Ile Leu Gln Met Arg
        1205 1210 1215
Asn Ser Lys Thr Gly Thr Glu Leu Asp Tyr Leu Ile Ser Pro Val
        1220 1225 1230
Ala Asp Val Asn Gly Asn Phe Phe Asp Ser Arg Gln Ala Pro Lys
        1235 1240 1245
Asn Met Pro Gln Asp Ala Asp Ala Asn Gly Ala Tyr His Ile Gly
        1250 1255 1260
Leu Lys Gly Leu Met Leu Leu Asp Arg Ile Lys Asn Asn Gln Glu
        1265 1270 1275
Gly Lys Lys Leu Asn Leu Val Ile Lys Asn Glu Glu Tyr Phe Glu
        1280 1285 1290
Phe Val Gln Asn Arg Asn Asn Ser ser Lys Ile
        1295 1300
```

1. A composition comprising (a) an mRNA encoding a Cpf1 polypeptide comprising SEQ ID NO: 1 or a variant thereof which has at least 60% identity to SEQ ID NO: 1, wherein the Cpf1 polypeptide comprises a RuvC-like domain and does not comprise an HNH domain, and (b) an engineered guide RNA comprising a sequence substantially complementary to a target nucleic acid sequence.

2. The composition of claim 1, wherein the Cpf1 polypeptide has at least 85% identity to SEQ ID NO: 1.

3. The composition of claim 1, wherein the Cpf1 polypeptide has at least 90% identity to SEQ ID NO: 1.

4. The composition of claim 1, wherein the Cpf1 polypeptide has at least 95% identity to SEQ ID NO: 1.

5. The composition of claim 1, wherein the Cpf1 polypeptide has at least 98% identity to SEQ ID NO: 1.

6. The composition of claim 1, wherein the Cpf1 polypeptide comprises an arginine-rich motif.

7. The composition of claim 1, wherein the Cpf1 polypeptide comprises a zinc finger-domain having a metal-binding site.

8. The composition of claim 1, wherein the Cpf1 polypeptide has nuclease activity.

9. The composition of claim 1, wherein the Cpf1 polypeptide is catalytically inactive.

10. The composition of claim 1, wherein an additional amino acid sequence is fused to the N- or C-terminus of the Cpf1 polypeptide.

11. The composition of claim 10, wherein the additional amino acid sequence has nucleic acid or chromatin modifying, transcription activating or transcription repressing activity.

12. The composition of claim 10, wherein an additional amino acid sequence comprises a nuclear localization signal.

13. The composition of claim 1, wherein the Cpf1 polypeptide comprises an amino acid sequence motif comprising residues 783-794, 784-794, 785-794, 786-794, 787-794, 788-794, 789-794, 783-793, 783-792, 783-791, 783-790, 783-789, 783-788, 784-793, 785-792, or 786-790 of SEQ ID NO: 1.

14. The composition of claim 1, wherein the Cpf1 polypeptide comprises at least one mutation compared to SEQ ID NO:1.

15. The composition of claim 1, wherein the composition is configured for intracellular delivery.


SEQUENCE LISTING (36 sequences)

SEQ ID NO: 1 - protein, 1304 aa, synthetic construct
  REGION 1..1304: note = Description of Artificial Sequence: Synthetic Cpf1 consensus sequence
  VARIANT 439: note = MOD_RES - Any naturally occurring amino acid
  VARIANT 504: note = MOD_RES - Any naturally occurring amino acid
  VARIANT 521: note = MOD_RES - Any naturally occurring amino acid
  VARIANT 539: note = MOD_RES - Any naturally occurring amino acid
  VARIANT 800: note = MOD_RES - Any naturally occurring amino acid
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSDDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQXLIAKKTEKAKYLSLETIKLALEEFNKHRDIDKQCRFEEILANFAAIPMIFDEIAQNKDNLAQISXKYQNQGKKDLLQASAEXDVKAIKDLLDQTNNLLHXLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGXPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNNSSKI

SEQ ID NO: 2 - protein, 5 aa, synthetic construct
  REGION 1..5: note = Description of Artificial Sequence: Synthetic Residues 786 - 791 of Cpf1 consensus sequence (SEQ ID NO: 1)
FQIYN

SEQ ID NO: 3 - protein, 6 aa, synthetic construct
  REGION 1..6: note = Description of Artificial Sequence: Synthetic Residues 786 - 791 of Cpf1 consensus sequence (SEQ ID NO: 1)
FQIYNK

SEQ ID NO: 4 - protein, 7 aa, synthetic construct
  REGION 1..7: note = Description of Artificial Sequence: Synthetic Residues 786 - 792 of Cpf1 consensus sequence (SEQ ID NO: 1)
FQIYNKD

SEQ ID NO: 5 - protein, 14 aa, synthetic construct
  REGION 1..14: note = Description of Artificial Sequence: Synthetic Residues 781 - 794 of Cpf1 consensus sequence (SEQ ID NO: 1)
  VARIANT 1: note = MOD_RES - Gly or Lys
  VARIANT 2: note = MOD_RES - Lys, Ser or Asp
  VARIANT 3: note = MOD_RES - Leu or Ile
  VARIANT 4: note = MOD_RES - Tyr or Phe
  VARIANT 5: note = MOD_RES - Leu or Met
  VARIANT 13: note = MOD_RES - Phe or Tyr
  VARIANT 14: note = MOD_RES - Ser, Ala or Val
XXXXXFQIYNKDXX

SEQ ID NO: 6 - protein, 14 aa, synthetic construct
  REGION 1..14: note = Description of Artificial Sequence: Synthetic Residues 781 - 794 of Cpf1 consensus sequence (SEQ ID NO: 1)
GKLYLFQIYNKDFS

SEQ ID NO: 7 - protein, 7 aa, synthetic construct
  REGION 1..7: note = Description of Artificial Sequence: Synthetic RuvC domain of catalytically inactive Cpf1
  VARIANT 1: note = MOD_RES - Gly or Ser
XIDRGER

SEQ ID NO: 8 - protein, 6 aa, synthetic construct
  REGION 1..6: note = Description of Artificial Sequence: Synthetic RuvC domain of catalytically inactive Cpf1
DANGAY

SEQ ID NO: 9 - skipped

SEQ ID NO: 10 - protein, 4 aa, synthetic construct
  REGION 1..4: note = Description of Artificial Sequence: Synthetic RuvC domain of catalytically inactive Cpf1
EDLN

SEQ ID NO: 11 - protein, 7 aa, synthetic construct
  REGION 1..7: note = Description of Artificial Sequence: Synthetic RuvC domain
SIDRGER

SEQ ID NO: 12 - protein, 7 aa, synthetic construct
  REGION 1..7: note = Description of Artificial Sequence: Synthetic RuvC domain
GIDRGER

SEQ ID NO: 13 - protein, 4 aa, synthetic construct
  REGION 1..4: note = Description of Artificial Sequence: Synthetic RuvC domain
ENLN

SEQ ID NO: 14 - protein, 4 aa, synthetic construct
  REGION 1..4: note = Description of Artificial Sequence: Synthetic RuvCdomain
EELN

SEQ ID NO: 15 - protein, 19 aa, synthetic construct
  REGION 1..19: note = Description of Artificial Sequence: Synthetic Arginine-rich motif
  VARIANT 1: note = MOD_RES - Asp or Asn
  VARIANT 3: note = MOD_RES - Arg, Gln or His
  VARIANT 4: note = MOD_RES - Lys, Glu, Ser or Asp
  VARIANT 5: note = MOD_RES - Ala, Lys or Leu
  VARIANT 7: note = MOD_RES - Asp, Asn or Ala
  VARIANT 8: note = MOD_RES - Val, Asn, Gln, Lys or Ala
  VARIANT 9: note = MOD_RES - Arg, Lys or Ile
  VARIANT 11: note = MOD_RES - Tyr, Lys or Ile
  VARIANT 12: note = MOD_RES - Asp or Glu
  VARIANT 13: note = MOD_RES - Asn, Arg or Met
  VARIANT 14: note = MOD_RES - Lys, Val, Phe or Asp
  VARIANT 15: note = MOD_RES - Glu, Ala, Asp or Ser
  VARIANT 18: note = MOD_RES - Arg, Gln or Lys
  VARIANT 19: note = MOD_RES - Asn, Ala, Ser or Asp
XYXXXLXXXEXXXXXARXX

SEQ ID NO: 16 - protein, 19 aa, synthetic construct
  REGION 1..19: note = Description of Artificial Sequence: Synthetic Arginine-rich motif
DYRKALDVREYDNKEARRN

SEQ ID NO: 17 - protein, 19 aa, synthetic construct
  REGION 1..19: note = Description of Artificial Sequence: Synthetic Arginine-rich motif
DYQKKLDNREKERVAARQA

SEQ ID NO: 18 - protein, 19 aa, synthetic construct
  REGION 1..19: note = Description of Artificial Sequence: Synthetic Arginine-rich motif
DYREKLNQREIEMKDARQS

SEQ ID NO: 19 - protein, 19 aa, synthetic construct
  REGION 1..19: note = Description of Artificial Sequence: Synthetic Arginine-rich motif
DYHSLLDKKEKERFEARQN

SEQ ID NO: 20 - protein, 19 aa, synthetic construct
  REGION 1..19: note = Description of Artificial Sequence: Synthetic Arginine-rich motif
NYHDKLAAIEKDRDSARKD

SEQ ID NO: 21 - DNA, 29 bp, Francisella tularensis
  source 1..29: mol_type = unassigned DNA; note = Francisella novicida
agattaaaaggtaattctatcttgttgag

SEQ ID NO: 22 - DNA, 45 bp, Francisella tularensis
  source 1..45: mol_type = unassigned DNA; note = Francisella novicida
ataatttaagattaaaaggtaattctattttgttgagatctgagc

SEQ ID NO: 23 - DNA, 26 bp, Francisella tularensis
  source 1..26: mol_type = unassigned DNA; note = Francisella novicida
tagcgatttatgaaggtcattttttt

SEQ ID NO: 24 - DNA, 42 bp, Francisella tularensis
  source 1..42: mol_type = unassigned DNA; note = Francisella novicida
ctaaattatagcgatttatgaaggtcatttttttaaaaagtt

SEQ ID NO: 25 - DNA, 29 bp, Francisella tularensis
  source 1..29: mol_type = unassigned DNA; note = Francisella novicida
atggattattacttaactggagtgtttac

SEQ ID NO: 26 - DNA, 45 bp, Francisella tularensis
  source 1..45: mol_type = unassigned DNA; note = Francisella novicida
aatgttcaatggattattacttaattggagtgtctacgtcgatgg

SEQ ID NO: 27 - DNA, 28 bp, Francisella tularensis
  source 1..28: mol_type = unassigned DNA; note = Francisella novicida
gccacaaatactacaaaaaataacttaa

SEQ ID NO: 28 - DNA, 44 bp, Francisella tularensis
  source 1..44: mol_type = unassigned DNA; note = Francisella novicida
attttttggctccaaatactacaaaaaataacttaaactttgaa

SEQ ID NO: 29 - DNA, 32 bp, Francisella tularensis
  source 1..32: mol_type = unassigned DNA; note = Francisella novicida
attgtcaaaacataagcagctgcttcaaatat

SEQ ID NO: 30 - DNA, 48 bp, Francisella tularensis
  source 1..48: mol_type = unassigned DNA; note = Francisella novicida
ggtcttttactgttattacataagcagccgcttcaaatatcttagcaa

SEQ ID NO: 31 - protein, 10 aa, synthetic construct
  REGION 1..10: note = Description of Artificial Sequence: Synthetic isolated polypeptide or fragment thereof having a nuclease activity
YLFQIYNKDF

SEQ ID NO: 32 - protein, 1184 aa, unidentified
  source 1..1184: note = Methanomethylophilus alvus
MDAKEFTGQYPLSKTLRFELRPIGRTWDNLEASGYLAEDRHRAECYPRAKELLDDNHRAFLNRVLPQIQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWK

SEQ ID NO: 33 - protein, 1265 aa, Acidaminococcus sp.
  source 1..1265: note = Acidaminococcus sp. BV3L6 8
MTQFEGFTNLYQVSKTLRFELIPQGKTLKHIQEQGFIEEDKARNDHYKELKPIIDRIYKTYADQCLQLVGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 34 - protein, 1187 aa, unidentified
  REGION 1..1187: note = Description of Unknown: Lachnospiraceae sequence
MSKLEKFTNCYSLSKTLRFKAIPVGKTQENIDNKRLLVEDEKRAEDYKGVKKLLDRYYLSFINDVLHSIGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 35 - protein, 1255 aa, Francisella tularensis
  source 1..1255: note = Francisella tularensis U112 1
MSIYQEFVNKYSLSKTLRFELIPQGKTLENIKARGLILDDEKRAKDYKKAKQIIDKYHQFFIEEILSSVDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 36 - protein, 1196 aa, unidentified
  source 1..1196: note = Candidatus Methanoplasma termitum 10
MNNYDEFTKLYPIQKTIRFELKPQGRTMEHLETFNFFEEDRDRAEKYKILKEAIDEYHKKFIDEHLTNMKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD